SEQUENCE LISTING (15 sequences)

SEQ ID NO: 1 - protein, 36 aa, Artificial Sequence
  peptide1
LRVRLASHLRKLRKRLLRDAKKKKKKKKKKKKKKKK

SEQ ID NO: 2 - protein, 36 aa, Artificial Sequence
  peptide2
KKKKKKKKKKKKKKKKLRVRLASHLRKLRKRLLRDA

SEQ ID NO: 3 - protein, 20 aa, Artificial Sequence
  peptide3
LRVRLASHLRKLRKRLLRDA

SEQ ID NO: 4 - protein, 13 aa, Artificial Sequence
  peptide4
GRKKRRQRRRPPQ

SEQ ID NO: 5 - protein, 15 aa, Artificial Sequence
  peptide5
RQIKIWFQNRRMKWK

SEQ ID NO: 6 - protein, 27 aa, Artificial Sequence
  peptide6
GWTLNSAGYLLGKINLKALAALAKKIL

SEQ ID NO: 7 - protein, 27 aa, Artificial Sequence
  peptide7
GALFLGFLGAAGSTMGAWSQPKKKRKV

SEQ ID NO: 8 - protein, 8 aa, Artificial Sequence
  peptide8
RRRRRRRR

SEQ ID NO: 9 - protein, 9 aa, Artificial Sequence
  peptide9
RRRRRRRRR

SEQ ID NO: 10 - protein, 10 aa, Artificial Sequence
  peptide10
RRRRRRRRRR

SEQ ID NO: 11 - protein, 16 aa, Artificial Sequence
  peptide11
HHHHHHHHHHHHHHHH

SEQ ID NO: 12 - protein, 18 aa, Artificial Sequence
  peptide12
LLIILRRRIRKQAHAHSK

SEQ ID NO: 13 - protein, 17 aa, Artificial Sequence
  peptide13
KLALKLALKALKAALKA

SEQ ID NO: 14 - protein, 18 aa, Artificial Sequence
  peptide14
LLIILRRRIRKQAHAHSK

SEQ ID NO: 15 - protein, 27 aa, Artificial Sequence
  peptide15
GWTLNSAGYLLGKINLKALAALAKKIL

(19) United States
(12) Patent Application Publication
(10) Pub. No.: US 2023/0144068 A1
(43) Pub. Date: May 11, 2023

(54) METHOD FOR PREPARING MODIFIED VECTOR AND METHOD FOR MODIFYING VECTOR

(71) Applicant: KANEKA CORPORATION, Osaka-shi, Osaka (JP)

(72) Inventors: Hirofumi MAEDA, Hyogo (JP); Takahide SASAKI, Hyogo (JP); Mitsuaki KITANO, Hyogo (JP)

(73) Assignee: KANEKA CORPORATION, Osaka-shi, Osaka (JP)

(21) Appl. No.: 17/915,311

(22) PCT Filed: Feb. 18, 2021
(86) PCT No.: PCT/JP2021/006113

§ 371 Date: Sep. 28, 2022

(30) Foreign Application Priority Data
Mar. 31, 2020 (JP) 2020-062936

(51) Int. Cl. C12N 15/861

(52) U.S. Cl. CPC C12N 15/861

(57) ABSTRACT

A method for preparing a modified vector and a method for modifying a vector, the methods including: binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and binding a functional group (C) in the ligand compound to the functional group (B), wherein the functional group (C) is capable of binding to the functional group (B) and the ligand compound includes the functional group (C).

### TECHNICAL FIELD

The present invention relates to a method for preparing a modified vector and a method for modifying a vector.

### BACKGROUND ART

Vectors have been widely used for gene therapy, but have a problem with, for example, vectors having insufficient transferability to target tissues.

In order to solve this problem, for example, proteins constituting vectors have been modified, but no effect enough to show drug efficacy has been obtained.

Therefore, in recent years, a method for chemically modifying a vector and introducing, into the vector, a ligand for enhancing the function has been developed (for example, NPL 1).

In this method, however, a reagent in which the site that binds to a functional group in a vector and the ligand for enhancing the function are integrated is used. Therefore, there are limitations on available reagents, and it is difficult to say that this method is an effective method.

As another method, a two-step chemical modification method has been reported (for example, PTL 1).

This method, however, cannot be easily said to be an easy method because it uses a cysteine residue and the cysteine residue needs to be isolated by a reduction treatment before chemical modification. In addition, this reduction treatment may cause the vector to be decomposed.

As another method, a method for modifying a vector to introduce a mutagenesis of a cysteine residue and then chemically modifying it has been reported (for example, NPL 2), but this method is not easily said to be an easy method because it takes time to introduce the mutagenesis.

Therefore, a method for preparing a modified vector and a method for modifying a vector that can easily introduce various ligands without modification or pretreatment of a vector are completely unknown, and providing these methods are strongly demanded.

### CITATION LIST

### Patent Literature

PTL 1: European Patent Application No. 3461836

### Non-Patent Literature

NPL 1: Biotechnol. Bioeng. 2005 92: 24-34

NPL 2: small 2013 9 3: 421-429

### SUMMARY OF INVENTION

### Technical Problem

The present invention solves the existing problems in the art and achieves the following object. That is, the object of the present invention is to provide a method for preparing a modified vector and a method for modifying a vector that can easily introduce various ligands without modification or pretreatment of a vector.

### Solution to Problem

As a result of diligent studies performed by the inventors of the present invention for the purpose of achieving the above object, the inventors found that a method for preparing a modified vector and a method for modifying a vector that can easily introduce various ligands without modification or pretreatment of a vector can be provided by using a method including: binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and binding a functional group (C) in the ligand compound to the functional group (B), wherein the functional group (C) is capable of binding to the functional group (B) and the ligand compound includes the functional group (C).

The present invention is based on the finding found by the inventors of the present invention, and means for solving the problems are as follows. That is,
<1> A method for preparing a modified vector, the method including:

binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and binding a functional group (C) in the ligand compound to the functional group (B), wherein the functional group (C) is capable of binding to the functional group (B) and the ligand compound includes the functional group (C).
<2> A method for modifying a vector, the method including:

binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and binding a functional group (C) in the ligand compound to the functional group (B), wherein the functional group (C) is capable of binding to the functional group (B) and the ligand compound includes the functional group (C).

### Advantageous Effects of Invention

According to the present invention, it is possible to solve the existing problems in the art, to achieve the following object, and to provide a method for preparing a modified vector and a method for modifying a vector that can easily introduce various ligands without modification or pretreatment of a vector.

### BRIEF DESCRIPTION OF DRAWINGS

FIG. 1 is a view showing the result of SDS-PAGE obtained after the first step in Example 1;

FIG. 2 is a view showing the result of SDS-PAGE obtained after the second step in Example 1;

FIG. 3 is a view showing the result of SDS-PAGE obtained after the first step and after the second step in Example 2 and Example 3;

FIG. 4 is a view showing the result of SDS-PAGE obtained after the first step and after the second step in Example 4;

FIG. 5 is a view showing a purification chart of cation exchange chromatography after the first step in Example 4; and

FIG. 6 is a view showing a purification chart of cation exchange chromatography after the second step in Example 4.

### DESCRIPTION OF EMBODIMENTS

### (Method for Preparing Modified Vector)

The method for preparing a modified vector includes: a first step of binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and a second step of binding a functional group (C) in the ligand compound to the functional group (B), wherein the functional group (C) is capable of binding to the functional group (B) and the ligand compound includes the functional group (C). The method can further include other steps.

The order of the first step and the second step is not particularly limited and may be appropriately selected depending on the intended purpose. The second step is preferably performed after the first step because various ligands capable of binding to the linker compound can be easily introduced into the vector and options of ligands are broadened.

### <First Step>

The first step is a step of binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound.

### —Vector—

The vector is not particularly limited and may be appropriately selected depending on the intended purpose. Examples thereof include virus vectors, plasmid vectors, artificial chromosome vectors, cosmid vectors, and fosmid vectors.

Among them, virus vectors are preferable because they are widely used for gene therapy.

A virus used as the virus vector is not particularly limited and may be appropriately selected depending on the intended purpose. Examples thereof include adeno-associated viruses (AAVs), adenoviruses, retroviruses, lentiviruses, herpesviruses, polioviruses, papillomaviruses, vaccinia viruses, and poxviruses.

Among them, adeno-associated viruses (AAVs) are preferable because of a low pathogenicity.

A serotype of the AAV used as the virus vector is not particularly limited and may be appropriately selected depending on the intended purpose. Examples thereof include AAV1 (type-1 AAV), AAV2 (type-2 AAV), AAV3 (type-3 AAV), AAV4 (type-4 AAV), AAV5 (type-5 AAV), AAV6 (type-6 AAV), AAV7 (type-7AAV), AAV8 (type-8 AAV), AAV9 (type-9 AAV), AAV10 (type-10 AAV), AAV11 (type-11 AAV), AAV12 (type-12 AAV), AAV13 (type-13 AAV), AAV14 (type-14 AAV), and variants thereof.

The variant is not particularly limited and may be appropriately selected depending on the intended purpose. Examples thereof include (wild-type AAV-) modified AAVs obtained by gene recombination in order to improve the tissue specificity of target cells (tropism of infected cells).

### ——Functional Group in Vector——

The functional group in the vector is not particularly limited and may be appropriately selected depending on the intended purpose. Examples thereof include an amino group, a guanidino group, a hydroxy group, a carboxyl group, and an indole group. Among them, an amino group is preferable.

The functional group in the vector may be a functional group in a lysine residue, an arginine residue, a tyrosine residue, a serine residue, a threonine residue, or a tryptophan residue. Among them, a functional group in a lysine residue is preferable.

When the vector is the adeno-associated virus (AAV), the functional group in the vector is preferably a functional group constituting a capsid of the adeno-associated virus.

The capsid consists of, for example, VP1, VP2, and VP3.

### —Linker Compound—

The linker compound includes functional group (A) capable of binding to the functional group in the vector and functional group (B) capable of binding to a ligand compound, and can further include a linkage part (D).

### ——Functional Group (A) Capable of Binding to Functional Group in Vector——

The functional group (A) capable of binding to the functional group in the vector is not particularly limited and may be appropriately selected depending on the intended purpose. Examples thereof include a succinimidyl group, an isocyanate group, an amino methoxyethyl group, a cyclohexene sulfonamide group, a carbonyl group, an aldehyde group, an unsaturated carbonyl group, a diazonium terephthalate, a halogen atom, a maleimidyl group, a phthalimidyl group, a diazobenzene group, an unsaturated nitrile group, an allenyl group, and a leaving group.

The leaving group represents OSO2R′ or OP(O)(OR′)₂, where R′ represents an alkyl group having from 1 through 6 carbon atoms; an aryl group having from 4 through 10 carbon atoms. Examples of the alkyl group having from 1 through 6 carbon atoms include straight-chain or branched alkyl groups. Among them, suitable examples thereof include: a methyl group; an ethyl group; (n-, i-) propyl groups; and (n-, i-, t-) butyl groups. Examples of the aryl group having from 4 through 10 carbon atoms include an aromatic hydrocarbon group or a heterocyclic group, composed of a monocyclic ring or fused rings, ring(s) being 5- or 6-membered, and the group being such as a phenyl group, (2-, 3-, 4-) tolyl groups, (1-, 2-) naphthyl groups, a 2-pyrrolyl group, a 2-furyl group, a 3-thienyl group, and a 2-pyridyl group. Examples thereof include a methanesulfonyl group and a toluenesulfonyl group.

Among the above, a succinimidyl group is preferable in terms of reactivity.

The number of the functional groups (A) may be one or may be two or more in one molecule of the linker compound.

### ——Functional Group (B) Capable of Binding to Ligand Compound——

The functional group (B) capable of binding to the ligand compound is not particularly limited and may be appropriately selected depending on the intended purpose. Examples thereof include an azido group, an alkynyl group, an alkenyl group, a carbonyl group, a phosphine group, a tetrazine group, a hydrazine group, and a hydroxylamine group.

Among them, an alkynyl group or an azido group is preferable, and an alkynyl group is more preferable, in terms of reactivity.

The number of the functional groups (B) may be one or may be two or more in one molecule of the linker compound. When the number of the functional groups (B) is two or more, it is not particularly limited and may be appropriately selected depending on the intended purpose. Examples thereof include an azido group and an alkynyl group.

### ——Linkage Part (D)——

The linkage part (D) is not particularly limited and may be appropriately selected depending on the intended purpose as long as the linkage part (D) can link the functional group (A) to the functional group (B) and does not react with the functional group (A), the functional group (B), and the ligand compound.

The linkage part (D) may have a straight-chain structure or a branched structure, but preferably has the straight-chain structure.

The linkage part (D) may be hydrophilic, hydrophobic, or amphipathic, but is preferably hydrophilic.

The length of the linkage part (D) is not particularly limited and may be appropriately selected depending on the intended purpose. For example, the length thereof can be adjusted depending on the number of carbon atoms constituting the linkage part (D).

The chemical structure of the linkage part (D) is not particularly limited and may be appropriately selected depending on the intended purpose. Examples thereof include a structure having an alkylene group, a structure having a carbonyl group, a structure having an ether bond, a structure having a carboxylate ester bond, and combinations thereof.

The structure having an ether bond may have a chain structure (a straight-chain such as ethylene glycol, and a branched chain such as propylene glycol), or may have a cyclic structure (a structure including, for example, tetrahydropyran, tetrahydrofuran, or 1,4-dioxane).

Specific examples of the linkage part (D) include an alkylene group, an alkyleneoxy group, and a poly(alkyleneoxy) group.

The alkylene group is not particularly limited and may be appropriately selected depending on the intended purpose. The alkylene group is preferably an alkylene group having from 1 through 20 carbon atoms, more preferably an alkylene group having from 1 through 10 carbon atoms, and still more preferably an alkylene group having from 1 through 4 carbon atoms.

The alkyleneoxy group is not particularly limited and may be appropriately selected depending on the intended purpose. The alkyleneoxy group is preferably an alkyleneoxy group having from 1 through 100 carbon atoms, more preferably an alkyleneoxy group having from 4 through 100 carbon atoms, and still more preferably an alkyleneoxy group having from 5 through 50 carbon atoms.

The poly(alkyleneoxy) group is not particularly limited and may be appropriately selected depending on the intended purpose. The poly(alkyleneoxy) group is preferably a poly(alkyleneoxy) group having from 1 through 100 carbon atoms, more preferably a poly(alkyleneoxy) group having from 5 through 50 carbon atoms, and still more preferably a poly(alkyleneoxy) group having from 6 through 40 carbon atoms.

An alkylene oxide unit in the poly(alkyleneoxy) group is not particularly limited and may be appropriately selected depending on the intended purpose. Preferable examples thereof include ethylene oxide and propylene oxide. Ethylene oxide is more preferable.

The linker compound is not particularly limited and may be appropriately selected depending on the intended purpose. Specific examples thereof include: DIBENZ[b,f]azocine-5(6H)-hexanoic acid, 11,12-didehydro-ε-oxo-,2,5-dioxo-3-sulfo-1-pyrrolidinyl ester, sodium salt; and 4,7,10,13,16-Pentaoxa-20-azatricosanoic acid, 23-(11,12-didehydrodibenz[b,f]azocin-5(6H)-yl)-19,23-dioxo-,2,5-dioxo-1-pyrrolidinyl ester.

—binding of Functional Group (A) to Functional Group in Vector—

Binding of the functional group (A) to the functional group in the vector is not particularly limited and may be appropriately selected depending on the intended purpose. Examples thereof include: a method in which a solution containing the linker compound and the vector is mixed and allowed to react; and a method in which a solution containing the linker compound bound to the ligand compound and the vector is mixed and allowed to react. A method in which a glycine solution is added after these methods and the resultant is further allowed to react may be performed.

A solvent of the solution may be an aqueous solvent or an organic solvent, or may be a mixed solvent of an aqueous solvent and an organic solvent.

The aqueous solvent is not particularly limited and may be appropriately selected depending on the intended purpose. Examples thereof include a HEPES (4-(2-hydroxyethyl)-1-piperazineethanesulfonic acid) buffer, a phosphate buffer, and distilled water.

The pH of the HEPES buffer or the phosphate buffer is not particularly limited and may be appropriately selected depending on the intended purpose. The pH is preferably pH 4 or more but pH 12 or less, more preferably pH 5 or more but pH 11 or less, still more preferably pH 6 or more but pH 10 or less, and particularly preferably pH 7 or more but pH 9 or less.

The organic solvent is not particularly limited and may be appropriately selected depending on the intended purpose. The organic solvent is preferably compatible with water. Examples thereof include: nitrile-based solvents such as acetonitrile; alcoholic solvents such as methanol, ethanol, and propanol; ether-based solvents such as tetrahydrofuran and 1,4-dioxane; sulfur-based solvents such as dimethyl sulfoxide; and amide-based solvents such as dimethylformamide.

A mixture ratio between the aqueous solvent and the organic solvent is not particularly limited unless insoluble components form significantly.

A final concentration of the linker compound (before addition of the glycine solution) is not particularly limited and may be appropriately selected depending on the intended purpose. The final concentration is preferably 0.1 μM or more but 200 mM or less, more preferably 0.5 μM or more but 100 mM or less, still more preferably 5 μM or more but 80 mM or less, and particularly preferably 10 μM or more but 50 mM or less.

A titer of the vector is not particularly limited and may be appropriately selected depending on the intended purpose. The titer is preferably 1×10⁴ vg/μL or more but 1×10¹⁰⁰ vg/μL or less, more preferably 1×10⁵ vg/μL or more but 5×10⁸⁰ vg/μL or less, and still more preferably 1×10⁶ vg/μL or more but 1×10⁵⁰ vg/μL or less.

A concentration of the glycine solution is not particularly limited and may be appropriately selected depending on the intended purpose. The concentration is preferably 0.1 μM or more but 20 M or less, more preferably 0.5 μM or more but 10 M or less, still more preferably 5 μM or more but 5 M or less, and particularly preferably 10 μM or more but 4 M or less.

A temperature of the reaction is not particularly limited and may be appropriately selected depending on the intended purpose. The temperature is preferably 0° C. or more but 70° C. or less, and more preferably 40° C. or less.

The reaction time before addition of the glycine solution is not particularly limited and may be appropriately selected depending on the intended purpose. The reaction time before addition of the glycine solution is preferably 5 minutes or more but 120 hours or less, more preferably 30 minutes or more but 96 hours or less, and still more preferably 1 hour or more but 72 hours or less.

The reaction time after addition of the glycine solution is not particularly limited and may be appropriately selected depending on the intended purpose. The reaction time after addition of the glycine solution is preferably 5 minutes or more but 120 hours or less, more preferably 30 minutes or more 96 hours or less, and still more preferably 1 hour or more but 72 hours or less.

### <Second Step>

The second step is a step of binding a functional group (C) in the ligand compound to the functional group (B), wherein the functional group (C) is capable of binding to the functional group (B) and the ligand compound includes the functional group (C).

### —Ligand Compound—

The ligand compound includes functional group (C) capable of binding to the functional group (B) and a ligand part, and can further include a linkage part (E).

The linkage part (E) is not particularly limited and may be appropriately selected depending on the intended purpose, as long as it can link the functional group (C) to the ligand part, and does not react with the linker compound, the functional group (C), and the ligand part.

The structure and specific examples of the linkage part (E) are the same as those of the linkage part (D) in the aforementioned linker compounds.

The ligand part in the ligand compound is a substance that has, for example, an affinity with, for example, tissues, cells, or proteins in organisms or a substance that uses an affinity to allow labeling. The ligand part is not particularly limited and may be appropriately selected depending on the intended purpose.

The term “labelling” means that functions such as membrane permeability and target cell specificity are added to the vector, and the phrase “functions are added” means modification. In a broad sense, it can be said that introduction of a linker is also considered as modification in terms of addition of the scaffold function that easily introduces other functional substances.

The ligand part is not particularly limited and may be appropriately selected depending on the intended purpose. Examples thereof include saccharide compounds, peptides, low-molecular weight compounds, and immunoglobulin.

The number of the ligand parts may be one or may be two or more in one molecule of the ligand compound. When the number of the ligand parts is two or more, it is not particularly limited and may be appropriately selected depending on the intended purpose. Examples thereof include saccharide compounds and peptides.

The saccharide compound is not particularly limited and may be appropriately selected depending on the intended purpose. Examples thereof include monosaccharides, oligosaccharides, and polysaccharides. Regarding the monosaccharide used herein, an asymmetric carbon may be an enantiomer. The oligosaccharide is formed of two to six molecules of monosaccharides. The polysaccharide is a polymer obtained by linking monosaccharides in a straight-chain manner or in a branched manner. In terms of easy availability, monosaccharides, disaccharides, and trisaccharides are preferable. The saccharide compound is not particularly limited and may be appropriately selected depending on the intended purpose. Examples thereof include allose, arabinose, erythrose, fructose, fucose, galactose, galactosamine, glucose, glucosamine, gulose, glucuronic acid, idose, inositol, lyxose, mannose, mannosamine, psicose, rhamnose, ribose, sialic acid, sorbose, tagatose, talose, xylose, disaccharide, trisaccharide, tetrasaccharide, oligosaccharide, cyclodextrin, 2-Azidoethyl 2-acetamido-2-deoxy-β-D-galactopyranoside, 2-Azidoethyl β-D-Glucopyranoside, and unnatural type sugars. Moreover, those obtained by adding polyethylene glycol groups or protecting groups to the aforementioned saccharide compounds may be used.

The protecting group is a substituent that protects a hydroxy group or an amino group in the saccharide compound and may be appropriately selected depending on the intended purpose. Examples of the protecting group of the hydroxy group include an acyl group, a carbonate group, a carbamate group, a cyclic acetal group, and an ether group. The acyl group is not particularly limited and may be appropriately selected depending on the intended purpose. Examples thereof include an acetyl group, a phenylacetyl group, a halogenated acetyl group, a methoxyacetyl group, a phenoxyacetyl group, a pivaloyl group, and a benzoyl group. The carbonate group is not particularly limited and may be appropriately selected depending on the intended purpose. Examples thereof include a t-butyl carbonate group and a benzyl carbonate group. The carbamate group is not particularly limited and may be appropriately selected depending on the intended purpose. Examples thereof include a phenyl carbamate group. The cyclic acetal group is not particularly limited and may be appropriately selected depending on the intended purpose. Examples thereof include a methylene acetal group, an ethylidene acetal group, an acetonide group, and a benzylidene acetal group. The ether group is not particularly limited and may be appropriately selected depending on the intended purpose. Examples thereof include a benzyl ether group.

Examples of the protecting group of the amino group include an acyl group and a carbamate group. The acyl group is not particularly limited and may be appropriately selected depending on the intended purpose. Examples thereof include an acetyl group, a phenylacetyl group, a halogenated acetyl group, a methoxyacetyl group, a phenoxyacetyl group, a pivaloyl group, and a benzoyl group. The carbamate group is not particularly limited and may be appropriately selected depending on the intended purpose. Examples thereof include a 9-fluorenyl methyl carbamate group, a phenyl carbamate group, and a t-butyl carbamate group.

Among these protecting groups, an acetyl group is preferable in terms of low toxicity and easy availability.

The peptide compound is not particularly limited and may be appropriately selected depending on the intended purpose. Examples thereof include

(SEQ ID NO: 1)
LRVRLASHLRKLRKRLLRDAKKKKKKKKKKKKKKKK,
(SEQ ID NO: 2)
KKKKKKKKKKKKKKKKLRVRLASHLRKLRKRLLRDA,
(SEQ ID NO: 3)
LRVRLASHLRKLRKRLLRDA,
(SEQ ID NO: 4)
GRKKRRQRRRPPQ,
(SEQ ID NO: 5)
RQIKIWFQNRRMKWK,
(SEQ ID NO: 6)
GWTLNSAGYLLGKINLKALAALAKKIL,
(SEQ ID NO: 7)
GALFLGFLGAAGSTMGAWSQPKKKRKV,
(SEQ ID NO: 8)
RRRRRRRR,
(SEQ ID NO: 9)
RRRRRRRRR,
(SEQ ID NO: 10)
RRRRRRRRRR,
(SEQ ID NO: 11)
HHHHHHHHHHHHHHHH,
(SEQ ID NO: 12)
LLIILRRRIRKQAHAHSK,
(SEQ ID NO: 13)
KLALKLALKALKAALKA,
(SEQ ID NO: 14)
LLIILRRRIRKQAHAHSK,
and
(SEQ ID NO: 15)
GWTLNSAGYLLGKINLKALAALAKKIL.

The peptide compound may have a cyclic structure. Moreover, each amino acid may be a D-amino acid, substituted and/or unnatural amino acids may be inserted into unnatural amino acids. The peptide compound may include a salt, and may be one obtained by adding a polyethylene glycol group thereto.

The low-molecular weight compound is not particularly limited and may be appropriately selected depending on the intended purpose. The low-molecular weight compound may be any compound as long as it exhibits drug efficacy in a living body. Examples thereof include Paclitaxel and low molecular antibodies. The low-molecular weight compound may be a substance for evaluating an affinity with a substance in a body. Examples thereof include biotin and dyes, and may be one obtained by adding a polyethylene glycol group thereto.

The dye is not particularly limited. Examples thereof include fluorescein, HiLyte Fluor 555, HiLyte Fluor 647, HiLyte Fluor 750, DyLight 350, DyLight 405, DyLight 550, DyLight 633, DyLight 755, Aleza Fluor 350, Aleza Fluor 405, Aleza Fluor 488, Aleza Fluor 532, Aleza Fluor 546, Aleza Fluor 555, Aleza Fluor 568, Aleza Fluor 594, Aleza Fluor 647, Aleza Fluor 680, Aleza Fluor 750, Cy 5, and Cy 3. Moreover, the dye may be one obtained by adding a polyethylene glycol group thereto.

The immunoglobulin is not particularly limited and may be appropriately selected depending on the intended purpose. Examples thereof include IgG, IgM, IgA, IgD, and IgE.

The upper limit of the weight average molecular weight of the ligand compound is not particularly limited and may be appropriately selected depending on the intended purpose. The upper limit is preferably 500,000 or less, more preferably 300,000 or less, still more preferably 200,000 or less, and particularly preferably 100,000 or less.

The lower limit of the weight average molecular weight of the ligand compound is not particularly limited and may be appropriately selected depending on the intended purpose. The lower limit is preferably 200 or more.

The ligand compound is not particularly limited and may be appropriately selected depending on the intended purpose. Specific examples thereof include 2-Azidoethyl 2-acetamido-2-deoxy-β-D-galactopyranoside and 2-Azidoethyl β-D-Glucopyranoside.

### ——Functional Group (C) Capable of Binding to Functional Group (B)——

The functional group (C) capable of binding to the functional group (B) is not particularly limited and may be appropriately selected depending on the intended purpose. When the functional group (B) is an azido group, a phosphine group or an alkynyl group is preferable. When the functional group (B) is an alkynyl group, an azido group is preferable. When the functional group (B) is an alkenyl group, a tetrazine group or an alkenyl group is preferable. When the functional group (B) is a carbonyl group, a hydrazine group or a hydroxylamine group is preferable. When the functional group (B) is a phosphine group, an azido group is preferable. When the functional group (B) is a tetrazine group, an alkenyl group is preferable. When the functional group (B) is a hydrazine group, a carbonyl group is preferable. When the functional group (B) is a hydroxylamine group, a carbonyl group is preferable.

The number of the functional groups (C) may be one or may be two or more in one molecule of the ligand compound. When the number of the functional groups (C) is two or more, it is not particularly limited and may be appropriately selected depending on the intended purpose. Examples thereof include an azido group and an alkynyl group.

### —Binding of Functional Group (C) to Functional Group (B)—

Binding of the functional group (C) to the functional group (B) is not particularly limited and may be appropriately selected depending on the intended purpose. Examples thereof include: a method in which a solution containing a reactant obtained by binding the functional group (A) to the functional group in the vector and the ligand compound is mixed and allowed to react; and a method in which a solution containing the linker compound and the ligand compound is mixed and allowed to react.

A solvent of the solution may be an aqueous solvent or an organic solvent, or may be a mixed solvent of an aqueous solvent and an organic solvent.

The aqueous solvent is not particularly limited and may be appropriately selected depending on the intended purpose. Examples thereof include a HEPES buffer, a phosphate buffer, and distilled water.

The pH of the HEPES buffer or the phosphate buffer is not particularly limited and may be appropriately selected depending on the intended purpose. The pH is preferably pH 1 or more but pH 12 or less, more preferably pH 2 or more but pH 12 or less, still more preferably pH 3 or more but pH 11 or less, and particularly preferably pH 4 or more but pH 10 or less.

The organic solvent is not particularly limited and may be appropriately selected depending on the intended purpose. The organic solvent is preferably compatible with water. Examples thereof include: nitrile-based solvents such as acetonitrile; alcoholic solvents such as methanol, ethanol, and propanol; ether-based solvents such as tetrahydrofuran and 1,4-dioxane; sulfur-based solvents such as dimethyl sulfoxide; and amide-based solvents such as dimethylformamide.

A mixture ratio between the aqueous solvent and the organic solvent is not particularly limited unless insoluble components form significantly.

A final concentration of the ligand compound is not particularly limited and may be appropriately selected depending on the intended purpose. The final concentration is preferably 0.01 mM or more but 100 mM or less, more preferably 0.1 mM or more but 80 mM or less, and still more preferably 0.2 mM or more but 50 mM or less.

A temperature of the reaction is not particularly limited and may be appropriately selected depending on the intended purpose. The temperature is preferably 0° C. or more but 70° C. or less, and more preferably 40° C. or less.

The reaction time is not particularly limited and may be appropriately selected depending on the intended purpose. The reaction time is preferably 5 minutes or more but 120 hours or less, more preferably 30 minutes or more 96 hours or less, and still more preferably 1 hour or more but 72 hours or less.

<Other Steps>

The other steps are not particularly limited and may be appropriately selected depending on the intended purpose. Examples thereof include a vector preparation step, a vector purification step, a reduction step, and a modified vector purification step.

### —Vector Preparation Step—

The vector preparation step is not particularly limited and may be appropriately selected depending on the intended purpose. Examples thereof include a method in which a gene encoding a vector is transfected into, for example, a culture cell and the vector is prepared from a cell lysate or a cell supernatant.

### —Vector Purification Step—

The vector purification step is not particularly limited and may be appropriately selected depending on the intended purpose. Examples thereof include: a purification method using a commercially available kit; and a method for purifying a cell lysate using, for example, cation chromatography, anion chromatography, size exclusion chromatography, a filter, or an ultrafiltration membrane.

### —Reduction Step—

The reduction step is not particularly limited and may be appropriately selected depending on the intended purpose. Examples thereof include: a step of adding a reducing agent such as DTT (dithiothreitol) to a reaction solution after the step of binding, to the functional group in the vector, the functional group (A) in the linker compound or after the step of binding, to the functional group (B), the functional group (C) in the ligand compound; and a step of adding a reducing agent such as DTT to a modified vector after the purification step.

### —Modified Vector Purification Step—

The modified vector purification step is not particularly limited and may be appropriately selected depending on the intended purpose. Examples thereof include: a purification method using, for example, cation exchange column chromatography, anion chromatography, size exclusion chromatography, a filter, or an ultrafiltration membrane. Purification may be performed between the first step and the second step of the modification reaction, or may be performed after the second step.

### (Method for Modifying Vector)

The method for modifying a vector includes: a first step of binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and a second step of binding a functional group (C) in the ligand compound to the functional group (B), wherein the functional group (C) is capable of binding to the functional group (B) and the ligand compound includes the functional group (C). The method can further include other steps.

The first step, the second step, and the other steps in the method for modifying a vector are as described in the method for preparing a modified vector.

### EXAMPLES

Examples of the present invention will be described hereinafter. However, the present invention should not be limited to these Examples.

### Reference Example 1: Preparation of AAV2

A medium solution of PEI (Polysciences), and a medium solution containing modified pAAV2 obtained by modifying pAAV2 (CELL BioLABs, Inc) with a fluorescent protein GFP so as to express VENUS (GenBank: ACQ43955.1), pRC-mi342 (Takara Bio Inc.), and pHelper (Takara Bio Inc.) were transfected into HEK293T cells (ATCC), and were cultured at 37° C. under 5% CO₂ for 4 days, to produce AAV2. Then, EDTA (Etylenediaminetetraacetic acid) was added thereto to exfoliate the cells. Then, the resultant was subjected to centrifugal separation, and cell pellet and a centrifugal supernatant were collected.

### Reference Example 2: Purification of AAV2

A vector was purified from the cell pellet obtained in Reference Example 1 using AAVpro (Registered Trademark) Purification Kit (AAV2) (TaKaRa 6232).

As a result of measuring the titer of the obtained purified AAV2 through RT-PCR (Quantstudio, SYBR Green method), the purified AAV2 was found to have a titer of 4×10⁹ vg/μL.

### Reference Example 3: Purification of AAV2

After a mixed solution of 40 wt % PEG8000 (Sigma) and 2.5 M NaCl aqueous solution was added to the centrifugal supernatant obtained in Reference Example 1, the resultant was left to stand at 4° C. for 16 hours. After centrifugal separation, a precipitation layer was treated with 0.1 v/v % TritonX-100-containing Dulbecco's Phosphate-Buffered Saline and a 1 M MgCl₂ aqueous solution, and was further treated with endonuclease. After the reaction was completed with EDTA, the resultant was subjected to centrifugal separation, and a supernatant was collected. The cell pellet obtained in Reference Example 1 was treated with 0.1 v/v % TritonX-100-containing Dulbecco's Phosphate-Buffered Saline and a 1 M MgCl₂ aqueous solution, and was further treated with endonuclease. After the reaction was completed with EDTA, the resultant was subjected to centrifugal separation, and a supernatant was collected. The obtained supernatants were mixed, and were purified through cation chromatography (Thermo, POROS 50HS) and anion chromatography (Thermo, POROS 50HQ). After concentration, AAV2 was obtained.

### Example 1: Modification of AAV2

The AAV2 solution obtained in Reference Example 2 was used.

### (Preparation of Buffer)

After a 1 M HEPES (gibco) aqueous solution, a 5 M NaCl (NACALAI TESQUE, INC.) aqueous solution, and Tween20 (Sigma) were each diluted with water, a 1 M NaOH aqueous solution was added thereto, to prepare a 50 mM HEPES aqueous solution, a 165 mM NaCl aqueous solution, and 0.1 v/v % Tween 20 aqueous solution, each of which had a pH of 8.3.

### (Preparation of Reaction Reagents 1A, 1B, 1C, 1D, 1E, 1F, and 1G)

The HEPES buffer prepared in the above manner was used to prepare reaction reagent 1A, which is a 0.125 mM DIBENZ[b,f]azocine-5(6H)-hexanoic acid,11,12-didehydro-ε-oxo-,2,5-dioxo-3-sulfo-1-pyrrolidinyl ester, sodium salt (Aldrich chemistry) solution. Then, it was subjected to 2-fold serial dilution, to prepare: a reaction reagent 1B, which is a 0.063 mM DIBENZ[b,f]azocine-5(6H)-hexanoic acid,11,12-didehydro-ε-oxo-,2,5-dioxo-3-sulfo-1-pyrrolidinyl ester, sodium salt solution; a reaction reagent 1C, which is a 0.032 mM DIBENZ[b,f]azocine-5(6H)-hexanoic acid,11,12-didehydro-ε-oxo-,2,5-dioxo-3-sulfo-1-pyrrolidinyl ester, sodium salt solution; a reaction reagent 1D, which is a 0.016 mM DIBENZ[b,f]azocine-5(6H)-hexanoic acid,11,12-didehydro-ε-oxo-,2,5-dioxo-3-sulfo-1-pyrrolidinyl ester, sodium salt solution; a reaction reagent 1E, which is a 0.008 mM DIBENZ[b,f]azocine-5(6H)-hexanoic acid,11,12-didehydro-ε-oxo-,2,5-dioxo-3-sulfo-1-pyrrolidinyl ester, sodium salt solution; a reaction reagent 1F, which is a 0.004 mM DIBENZ[b,f]azocine-5(6H)-hexanoic acid,11,12-didehydro-ε-oxo-,2,5-dioxo-3-sulfo-1-pyrrolidinyl ester, sodium salt solution; and a reaction reagent 1G, which is a 0.002 mM DIBENZ[b,f]azocine-5(6H)-hexanoic acid,11,12-didehydro-ε-oxo-,2,5-dioxo-3-sulfo-1-pyrrolidinyl ester, sodium salt solution.

### (Preparation of Reaction Reagents 2A, 2B, 2C, and 2D)

The HEPES buffer prepared in the above manner was used to prepare reaction reagent 2A, which is a 16 mM 2-Azidoethyl 2-acetamido-2-deoxy-β-D-galactopyranoside (Sigma-Aldrich) solution. Then, it was subjected to 4-fold serial dilution, to prepare: reaction reagent 2B, which is a 4 mM 2-Azidoethyl 2-acetamido-2-deoxy-β-D-galactopyranoside solution; reaction reagent 2C, which is a 1 mM 2-Azidoethyl 2-acetamido-2-deoxy-β-D-galactopyranoside solution; and reaction reagent 2D, which is a 0.25 mM 2-Azidoethyl 2-acetamido-2-deoxy-β-D-galactopyranoside solution.

### (First Step)

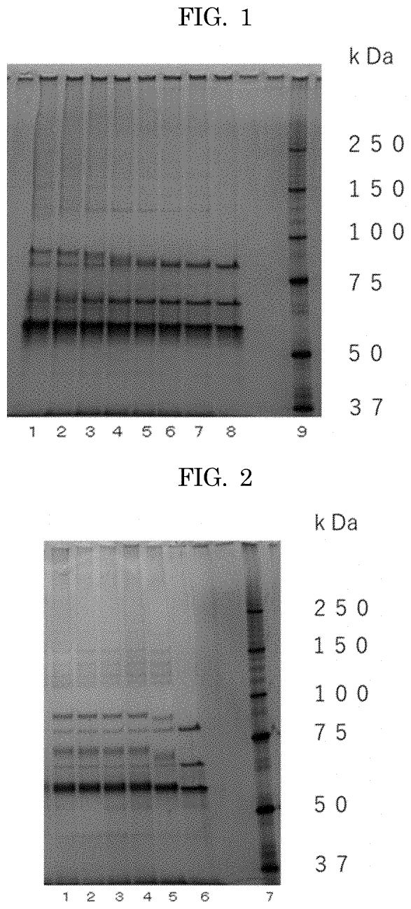

Each of the reaction reagents 1A, 1B, 1C, 1D, 1E, 1F, and 1G (9 μL) and the purified AAV2 solution (1 μL) prepared in the above manners were mixed and were allowed to react in an incubator at 37° C. for 16 hours. A 2M glycine solution (Wako) (2 μL) prepared with the above buffer was further added thereto, and was allowed to react in an incubator at 37° C. for 5 hours, to obtain first step product 1A, first step product 1B, first step product 1C, first step product 1D, first step product 1E, first step product 1F, and first step product 1G. After the reaction solution was reduced with a DTT-containing sample buffer, SDS-PAGE (ATTO) was performed, and silver staining (Invitrogen) was used to confirm the progress of reaction. The result of silver staining was shown in FIG. 1. The band of VP1 was about 82 kDa before reaction, and was about 88 kDa after reaction. Therefore, a compound was found to be added by 8 kDa.

In FIG. 1, lane 1 is a sample of the first step product 1A, lane 2 is a sample of the first step product 1B, lane 3 is a sample of the first step product 1C, lane 4 is a sample of the first step product 1D, lane 5 is a sample of the first step product 1E, lane 6 is a sample of the first step product 1F, lane 7 is a sample of the first step product 1G, lane 8 is a sample of the unmodified AAV2, and lane 9 is a marker.

### (Second Step)

Each of the reaction reagents 2A, 2B, 2C, and 2D (24 μL) and the above reaction solution of the first step product 1A (12 μL) prepared in the above manners were mixed and were allowed to react in an incubator at 37° C. for 16 hours, to obtain modified vector 2A, modified vector 2B, modified vector 2C, and modified vector 2D. After the reaction solution was reduced with a DTT (DITHIOTHREITOL)-containing sample buffer, SDS-PAGE (ATTO) was performed, and silver staining (Invitrogen) was used to confirm the progress of reaction. The result of silver staining was shown in FIG. 2. The band of VP1 was about 88 kDa before reaction, and was about 92 kDa after reaction. Therefore, a compound was found to be added by 4 kDa.

In FIG. 2, lane 1 is a sample of the modified vector 2A, lane 2 is a sample of the modified vector 2B, lane 3 is a sample of the modified vector 2C, lane 4 is a sample of the modified vector 2D, lane 5 is a sample obtained by using a buffer instead of the reaction reagent 2, lane 6 is the unmodified AAV2, and lane 7 is a marker.

### Example 2: Modification of AAV2

The purified AAV2 solution obtained in Reference Example 2 was used.

### (Preparation of Phosphate Buffer)

A 200 mM sodium dihydrogenphosphate dehydrate (Wako) and a 200 mM disodium hydrogenphosphate dodecahydrate (Wako) were mixed and diluted to obtain a 20 mM phosphate buffer having a pH of 8.5.

### (Preparation of Reaction Reagent 1H)

The phosphate buffer prepared in the above manner was used to prepare a 2 mM DIBENZ[b,f]azocine-5(6H)-hexanoic acid, 11,12-didehydro-ε-oxo-,2,5-dioxo-3-sulfo-1-pyrrolidinyl ester, sodium salt (Aldrich) solution (reaction reagent 1H).

### (Preparation of Reaction Reagent 3)

The phosphate buffer prepared in the above manner was used to prepare a 16 mM 2-Azidoethyl β-D-Glucopyranoside (TCI) solution (reaction reagent 3).

### (First Step)

The reaction reagent 1H (9 μL) and the purified AAV2 solution (1 μL) prepared in the above manners were mixed and were allowed to react in an incubator at 37° C. for 16 hours. A 2M glycine solution (Wako) (2 μL) prepared with the above phosphate buffer was further added thereto, and was left to stand in an incubator at 37° C. for 6 hours to stop the reaction, to obtain first step product 1H.

### (Second Step)

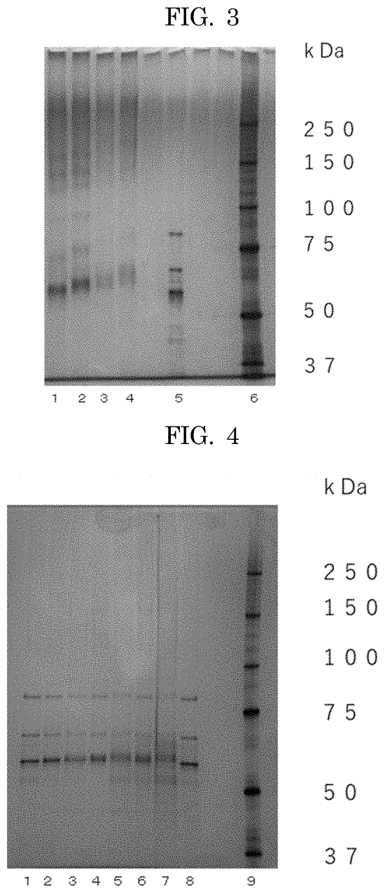

The reaction reagent 3 was allowed to react in the same manner as in Example 1, to obtain modified vector 3A. SDS-PAGE (ATTO, E-R7.5L) was performed, and silver staining (Invitrogen) was used to confirm the progress of reaction. The result of silver staining was shown in FIG. 3. The band of VP3 was about 60 kDa before reaction, and was about 62 kDa after reaction. Therefore, a compound was found to be added by 2 kDa.

In FIG. 3, lane 1 is a sample of the first step product 1H of Example 2 (reaction reagent 1H), lane 2 is a sample of the modified vector 3A of Example 2 (reaction reagent 1H), and lane 5 is a sample of the unmodified AAV2, and lane 6 is a marker.

### Example 3: Modification of AAV2

The purified AAV2 solution obtained in Reference Example 2 was used.

### (Preparation of Reaction Reagent 4)

The phosphate buffer prepared in the above manner was used to prepare a 2 mM 4,7,10,13,16-Pentaoxa-20-azatricosanoic acid, 23-(11,12-didehydrodibenz[b,f]azocin-5(6H)-yl)-19,23-dioxo-,2,5-dioxo-1-pyrrolidinyl ester (BROADPHARM) solution (reaction reagent 4).

### (First Step)

The reaction reagent 4 (9 μL) and the purified AAV2 solution (1 μL) prepared in the above manners were mixed and were allowed to react in an incubator at 37° C. for 16 hours. A 2M glycine solution (Wako) (2 μL) prepared with the above phosphate buffer was further added thereto, and was left to stand in an incubator at 37° C. for 6 hours to stop the reaction, to obtain first step product 4. After the reaction solution was reduced with a DTT-containing sample buffer, SDS-PAGE (ATTO) was performed, and silver staining (Invitrogen) was used to confirm the progress of reaction. The result of silver staining was shown in FIG. 3. The band of VP3 was about 60 kDa before reaction, and was about 64 kDa after reaction. Therefore, a compound was found to be added by 4 kDa.

### (Second Step)

The reaction reagent 3 was allowed to react in the same manner as in Example 1, to obtain modified vector 3B. SDS-PAGE (ATTO, E-R7.5L) was performed, and silver staining (Invitrogen) was used to confirm the progress of reaction. The result of silver staining was shown in FIG. 3. The band of VP3 was about 64 kDa before reaction, and was about 68 kDa after reaction. Therefore, a compound was found to be added by 4 kDa.

In FIG. 3, lane 3 is a sample of the first step product 4 of Example 3 (reaction reagent 4), lane 4 is a sample of the modified vector 3B of Example 3 (reaction reagent 4), lane 5 is a sample of the unmodified AAV2, and lane 6 is a marker.

### Example 4: Modification of AAV2

The AAV2 solution (6.6×10⁹ vg/μL, 500 μL) obtained in Reference Example 3 was used.

### (Preparation of Reaction Reagents 1I and 1J)

The same HEPES buffer as that of Example 1 was used to prepare a 1 mM DIBENZ[b,f]azocine-5(6H)-hexanoic acid, 11,12-didehydro-ε-oxo-,2,5-dioxo-3-sulfo-1-pyrrolidinyl ester, sodium salt (Aldrich chemistry) solution; i.e., reaction reagent 1I. The reaction reagent 1I was diluted to prepare a 0.5 mM solution, a reaction reagent 1J.

### (Preparation of Reaction Reagent 2E)

The same HEPES buffer as that of Example 1 was used to prepare a 2 mM 2-Azidoethyl 2-acetamido-2-deoxy-β-D-galactopyranoside (Sigma-Aldrich) solution; i.e., reaction reagent 2E.

### (First Step)

Each of the reaction reagents 1 (1I, 1J, and 1A) (4.5 mL) prepared in the above manner and Example 1 and the AAV2 solution (0.5 mL) were mixed and were allowed to react in an incubator at 37° C. for 16 hours. A 2M glycine solution (Wako) (1 mL) prepared with the same HEPES buffer as that of Example 1 was further added to the reaction solution, and was allowed to react in an incubator at 37° C. for 7 hours, to obtain first step product 1I, first step product 1J, and first step product 1A. After the reaction solution was reduced with a DTT-containing sample buffer, SDS-PAGE (ATTO E-R7.5L) was performed, and silver staining (Invitrogen 45-1001) was used to confirm the progress of reaction. The result of silver staining was shown in FIG. 4. The band of VP3 was about 60 kDa before reaction, and was about 62 kDa after reaction. Therefore, a compound was found to be added by 2 kDa.

In FIG. 4, lane 1 and lane 8 are the unmodified AAV2, lane 2 is a sample of the first step product 1A of Example 4 (reaction reagent 1A: 0.125 mM), lane 4 is a sample of the first step product 1J of Example 4 (reaction reagent 1J: 0.5 mM), lane 6 is a sample of the first step product 1I of Example 4 (reaction reagent 1I: 1 mM), and lane 9 is a marker.

### (Purification of Modified AAV2)

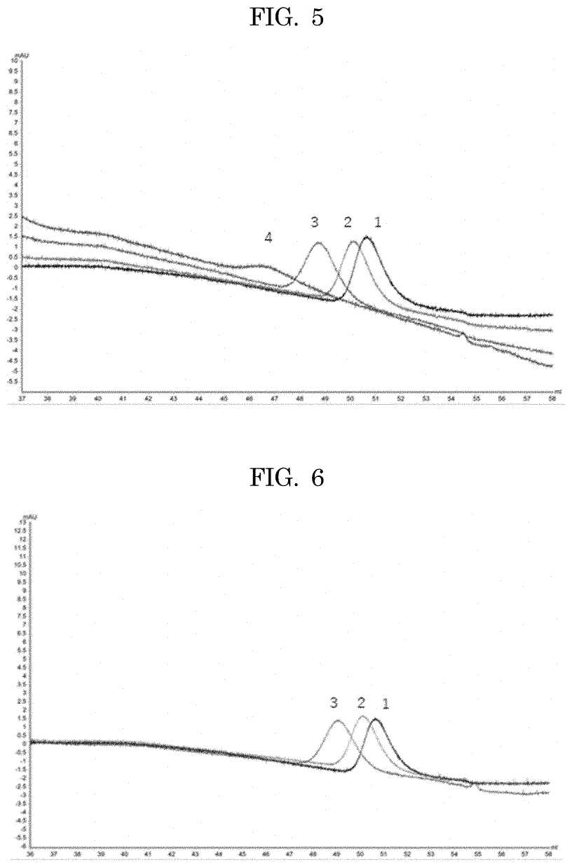

The first step product 1I, the first step product 1J, and the first step product 1A obtained by reacting with the reaction reagent 1I, the reaction reagent 1J, and the reaction reagent 1A, respectively in Example 4 were purified through cation exchange column chromatography (carrier: POROS 50HS, eluent: 20 mM phosphate buffer (pH 7.4), gradient condition: 100 mM to 370 mM NaCl aqueous solutions). The result was shown in FIG. 5. As a result of measuring the titers through RT-PCR (Quantstudio, SYBR Green method) after purification, the first step product 1I was found to have a titer of 5.8×10¹¹ vg/μL, the first step product 1J was found to have a titer of 1.8×10¹² vg/μL, and the first step product 1A was found to have a titer of 1.2×10¹² vg/μL.

In FIG. 5, the number 1 represents the unmodified AAV2, the number 2 represents a sample of the first step product 1A of Example 4 (reaction reagent 1A: 0.125 mM), the number 3 represents a sample of the first step product 1J of Example 4 (reaction reagent 1J: 0.5 mM), and the number 4 represents a sample of the first step product 1I Example 4 (reaction reagent 1I: 1 mM). In FIG. 5, the longitudinal axis represents absorbance at 280 nm, and the horizontal axis represents elution volume.

### (Second Step)

To the reaction solution (3 mL) obtained after the above reaction, each of the reaction reagents 2E, 2C, and 2D (6 mL) was added and was mixed. The resultant was allowed to react in an incubator at 37° C. for 16 hours, to obtain modified vector 2E, modified vector 2F, and modified vector 2G, respectively. After the reaction solution was reduced with a DTT-containing sample buffer, SDS-PAGE (ATTO) was performed, and silver staining (Invitrogen) was used to confirm the progress of reaction. The result of silver staining was shown in FIG. 4. The band of VP3 was about 62 kDa before reaction, and was about 63 kDa after reaction. Therefore, a compound was found to be added by 1 kDa.

In FIG. 4, lane 1 and lane 8 are the unmodified AAV2, lane 3 is a sample of the modified vector 2G of Example 4 (reaction reagent 1A: 0.125 mM and reaction reagent 2D: 0.25 mM), lane 5 is a sample of the modified vector 2F of Example 4 (reaction reagent 1J: 0.5 mM and reaction reagent 2C: 1 mM), lane 7 is a sample of the modified vector 2E of Example 4 (reaction reagent 1I: 1 mM and reaction reagent 2E: 2 mM), and lane 9 is a marker.

### (Purification of Modified Vectors 2G and 2F)

The modified vector 2F and the modified vector 2G obtained by reacting with the reaction reagent 2C and the reaction reagent 2D, respectively in Example 4 were purified through cation exchange column chromatography (carrier: POROS 50HS, eluent: 20 mM phosphate buffer (pH 7.4), gradient condition: 100 mM to 370 mM NaCl aqueous solutions). The result was shown in FIG. 6. As a result of measuring the titers through RT-PCR (Quantstudio, SYBR Green method) after purification, the modified vector 2F was found to have a titer of 2.6×10¹² vg/μL, and the modified vector 2G was found to have a titer of 2.6×10¹² vg/μL.

In FIG. 6, the number 1 represents the unmodified AAV2, the number 2 represents a sample of the modified vector 2G of Example 4 (reaction reagent 1A: 0.125 mM and reaction reagent 2D: 0.25 mM), the number 3 represents a sample of the modified vector 2F of Example 4 (reaction reagent 1J: 0.5 mM and reaction reagent 2C: 1 mM). In FIG. 6, the longitudinal axis represents absorbance at 280 nm, and the horizontal axis represents elution volume.

### (Purification of Modified Vector 2E)

The modified AAV2 obtained by reacting with the reaction reagent 2E in Example 4 was subjected to centrifugal concentration five times using an ultrafiltration membrane obtained from GE Healthcare (Vivaspin 20 kilograms to 100 kilograms). Finally, it was purified through buffer substitution to a 20 mM phosphate buffer (pH: 7.4, containing 250 mM NaCl). As a result of measuring the titer through RT-PCR (Quantstudio, SYBR Green method) after purification, the modified vector 2E was found to have a titer of 1.8×10¹² vg/μL.

Aspects of the present invention are as follows, for example.
<1> A method for preparing a modified vector, the method including:

binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and

binding a functional group (C) in the ligand compound to the functional group (B), wherein the functional group (C) is capable of binding to the functional group (B) and the ligand compound includes the functional group (C).
<2> The method according to <1>,

wherein the functional group in the vector is an amino group, a guanidino group, a hydroxy group, a carboxyl group, or an indole group.
<3> The method according to <1> or <2>,

wherein the functional group in the vector is a functional group in a lysine residue, an arginine residue, a tyrosine residue, a serine residue, a threonine residue, or a tryptophan residue.
<4> The method according to any one of <1> to <3>,

wherein the functional group (A) is a succinimidyl group.
<5> The method according to any one of <1> to <4>,

wherein the functional group (B) is an azido group, an alkynyl group, an alkenyl group, a carbonyl group, a phosphine group, a tetrazine group, a hydrazine group, or a hydroxylamine group.
<6> The method according to any one of <1> to <5>,

wherein a weight average molecular weight of the ligand compound is 100,000 or less.
<7> The method according to any one of <1> to <6>,

wherein the ligand compound is a saccharide compound.
<8> The method according to <7>,

wherein the saccharide compound includes a polyethylene glycol group.
<9> The method according to any one of <1> to <6>,

wherein the ligand compound is a peptide.
<10> The method according to any one of <1> to <9>,

wherein the linker compound includes a polyethylene glycol group.
<11> The method according to any one of <1> to <10>,

wherein the vector is an adeno-associated virus vector.
<12> The method according to <11>,

wherein the functional group in the vector is a functional group constituting a capsid of the adeno-associated virus vector.
<13> The method according to any one of <1> to <12>,

wherein the functional group in the vector is an amino group, the functional group (A) is a succinimidyl group, the functional group (B) or the functional group (C) is an azido group and/or an alkynyl group, and the ligand compound is a saccharide compound and/or a peptide.
<14> The method according to any one of <1> to <12>,

wherein the functional group in the vector is a functional group in a lysine residue, the functional group (B) or the functional group (C) is an azido group and/or an alkynyl group, and the ligand compound is a saccharide compound and/or a peptide.
<15> A method for modifying a vector, the method including:

binding a functional group (A) in a linker compound to a functional group in a vector, wherein the functional group (A) is capable of binding to the functional group, the linker compound includes the functional group (A) and a functional group (B), and the functional group (B) is capable of binding to a ligand compound; and

binding a functional group (C) in the ligand compound to the functional group (B), wherein the functional group (C) is capable of binding to the functional group (B) and the ligand compound includes the functional group (C).

1. A method for preparing a modified vector, the method comprising:
binding a functional group (A) in a linker compound to a functional group in a vector, wherein the functional group (A) is capable of binding to the functional group in the vector, the linker compound comprises the functional group (A) and a functional group (B), and the functional group (B) is capable of binding to a ligand compound; and
binding a functional group (C) in the ligand compound to the functional group (B), wherein the functional group (C) is capable of binding to the functional group (B) and the ligand compound comprises the functional group (C).

2. The method according to claim 1,
wherein the functional group in the vector is an amino group, a guanidino group, a hydroxy group, a carboxyl group, or an indole group.

3. The method according to claim 1,
wherein the functional group in the vector is a functional group in a lysine residue, an arginine residue, a tyrosine residue, a serine residue, a threonine residue, or a tryptophan residue.

4. The method according to claim 1,
wherein the functional group (A) is a succinimidyl group.

5. The method according to claim 1,
wherein the functional group (B) is an azido group, an alkynyl group, an alkenyl group, a carbonyl group, a phosphine group, a tetrazine group, a hydrazine group, or a hydroxylamine group.

6. The method according to claim 1,
wherein a weight average molecular weight of the ligand compound is 100,000 or less.

7. The method according to claim 1,
wherein the ligand compound is a saccharide compound.

8. The method according to claim 7,
wherein the saccharide compound includes a polyethylene glycol group.

9. The method according to claim 1,
wherein the ligand compound is a peptide.

10. The method according to claim 1,
wherein the linker compound includes a polyethylene glycol group.

11. The method according to claim 1,
wherein the vector is an adeno-associated virus vector.

12. The method according to claim 11,
wherein the functional group in the vector is a functional group constituting a capsid of the adeno-associated virus vector.

13. The method according to claim 1,
wherein the functional group in the vector is an amino group, the functional group (A) is a succinimidyl group, the functional group (B) or the functional group (C) is an azido group and/or an alkynyl group, and the ligand compound is a saccharide compound and/or a peptide.

14. The method according to claim 1,
wherein the functional group in the vector is a functional group in a lysine residue, the functional group (B) or the functional group (C) is an azido group and/or an alkynyl group, and the ligand compound is a saccharide compound and/or a peptide.

15. A method for modifying a vector, the method comprising:
binding a functional group (A) in a linker compound to a functional group in a vector, wherein the functional group (A) is capable of binding to the functional group in the vector, the linker compound comprises the functional group (A) and a functional group (B), and the functional group (B) is capable of binding to a ligand compound; and
binding a functional group (C) in the ligand compound to the functional group (B), wherein the functional group (C) is capable of binding to the functional group (B) and the ligand compound comprises the functional group (C).

16. The method according to claim 1, wherein:
the vector is an adeno-associated virus vector;
the functional group in the vector is an amino group, a guanidino group, a hydroxy group, a carboxyl group, an indole group, a lysine residue, an arginine residue, a tyrosine residue, a serine residue, a threonine residue, or a tryptophan residue;
the functional group (A) is a succinimidyl group, an isocyanate group, an amino methoxyethyl group, a cyclohexene sulfonamide group, a carbonyl group, an aldehyde group, an unsaturated carbonyl group, a diazonium terephthalate, a halogen atom, a maleimidyl group, a phthalimidyl group, a diazobenzene group, an unsaturated nitrile group, or an allenyl group;
the functional group (B) is an azido group, an alkynyl group, an alkenyl group, a carbonyl group, a phosphine group, a tetrazine group, a hydrazine group, or a hydroxylamine group;
the functional group (C) is a phosphine group, an alkynyl group, an azido group, a tetrazine group, an alkenyl group, a hydrazine group, a hydroxylamine group or a carbonyl group; and
the ligand compound is a saccharide compound or a peptide.

17. The method according to claim 16, wherein:
the functional group in the vector is an amino group;
the functional group (A) is a succinimidyl group;
the functional group (B) is an azido group or an alkynyl group; and
the functional group (C) is a phosphine group or an alkynyl group when said function group (B) is an azido group, and the functional group (C) is an azido group when said functional group (B) is an alkynyl group.

18. The method according to claim 17, wherein:
the functional group in the vector is a functional group in a lysine residue; and
the ligand compound is a saccharide compound.

19. The method according to claim 1, wherein:
the linker compound is DIBENZ[b,f]azocine-5(6H)-hexanoic acid, 11,12-didehydro-ε-oxo-,2,5-dioxo-3-sulfo-1-pyrrolidinyl ester, sodium salt, or 4,7,10,13,16-Pentaoxa-20-azatricosanoic acid, 23-(11,12-didehydrodibenz[b,f]azocin-5(6H)-yl)-19,23-dioxo-,2,5-dioxo-1-pyrrolidinyl ester.
